(19) United States
(12) Patent Application Publication
(10) Pub. No.: US 2022/0133262 A1
(43) Pub. Date: May 5, 2022

(54) ULTRASOUND TRANSDUCER ASSEMBLY, PROBE, ENDOSCOPY SYSTEM AND MANUFACTURING METHOD

(71) Applicant: The Hong Kong Polytechnic University, Hong Kong (CN)

(72) Inventors: Kwok-ho LAM, Hong Kong (CN); Riqiang LIN, Hong Kong (CN)

(21) Appl. No.: 17/249,873

(22) Filed: Mar. 17, 2021

(30) Foreign Application Priority Data
Oct. 29, 2020 (CN) 202011176786.4

(51) Int. Cl. A61B 8/12; B06B 1/06; A61B 8/00

(52) U.S. Cl. CPC A61B 8/12; B06B 2201/70; A61B 8/445; B06B 1/06

(57) ABSTRACT

The present disclosure provides an ultrasound transducer (UT) assembly for ultrasonic and photoacoustic dual-mode imaging of an endoscope, including a UT and a microlens integrated in the UT. The microlens is used for beam collimation or focusing and accommodated in an aperture of the UT. A probe/catheter including the UT assembly, an endoscopy system including the probe/catheter, and a method of manufacturing the UT assembly are also provided. The present application uses a photocurable glue and mold to form the microlens integrated in the UT and adopts the coaxial arrangement of the devices to solve the problems of light supply and device dimensions (rigid length and diameter), thereby simplifying the manufacturing process of intravascular photoacoustic (IVPA) probe/catheter.

### CROSS-REFERENCE TO RELATED APPLICATIONS

This application claims the benefit of China Patent Application No. 202011176786.4, filed on Oct. 29, 2020, which is incorporated by reference herein in its entirety.

### TECHNICAL FIELD

The present invention relates to an ultrasound transducer (UT) assembly for ultrasonic and photoacoustic dual-mode imaging of an endoscope, an ultrasonic and photoacoustic probe including the assembly, an endoscopy system including the ultrasonic and photoacoustic probe, and a method for manufacturing the UT assembly.

### BACKGROUND

Rupture of vulnerable plaque is the main cause of acute cardiovascular events. Early diagnosis and early warning of vulnerable plaque is one of the key technical means to reduce the mortality of cardiovascular diseases. There are three existing clinical intravascular imaging technologies, i.e., intravascular ultrasound, intravascular optical coherence tomography (OCT), and intravascular infrared spectroscopy. Intravascular ultrasonic imaging technology can distinguish the structure of each layer of a blood vessel wall, but ultrasonic imaging technology cannot accurately determine the plaque composition due to the close acoustic impedance of each soft tissue composition. Intravascular optical tomography technology reaches a high resolution of 10-20 μm, which allows accurate detection of a thin fiber cap on the plaque. However, its imaging depth is usually about 1 mm only, and the imaging depth on the plaque is even smaller, it therefore cannot evaluate the overall structure of the plaque. Intravascular infrared spectroscopy can obtain tissue composition information, but without depth information, which causes the physical location of the composition cannot be acquired. Therefore, it has become an urgent demand for clinical applications to research and develop an intravascular imaging system that can obtain morphological and component information with high resolution and large imaging depth.

Intravascular photoacoustic (IVPA) imaging technology is a type of intravascular imaging technology for atherosclerosis, which exhibits a great potential in acquiring plaque tissue composition, morphology of the vasa vasorum and inflammation physiological function information. The underlying principle of photoacoustic imaging is to obtain information on tissue light absorption by probing an ultrasonic signal (photoacoustic signal) generated due to the instantaneous thermoelastic effect after a biological tissue absorbs pulsed laser. Contrast of photoacoustic imaging is derived from light absorption, and imaging depth is mainly derived from the ultrasonic signal, which makes photoacoustic imaging fundamentally break through limitations on low penetration depth of high-resolution pure optical imaging methods such as OCT and confocal microscope due to light scattering. Based on the selective light absorption of different molecules and photo-photoacoustic spectroscopy methods, high-sensitivity detection of plaque chemical components can be achieved. Photoacoustic imaging technology itself can obtain depth information of the tissue, and in combination with ultrasound imaging technology, it can distinguish the structure of each layer of the blood vessel wall and the distribution of plaques, providing a powerful basis for judgment and identification of vulnerable plaques.

IVPA endoscopic catheter/probe is a key tool for photoacoustic imaging of plaques and vasa vasorum. Its main designs include two types as follows:

1. The optical element is placed next to a UT. For example, methods as adopted is described in Chinese Patents Nos. 201410829245.5, 201710846057.7, and 201810121955.0. Optical elements, such as optical fibers, gradient index (GRIN) lenses, and mirrors, are all positioned in a straight line, while UT is placed on the side or top of the optical element.

In photoacoustic endoscopy, a higher light fluence will result in higher photoacoustic signals. In order to provide better imaging effects, most catheters use GRIN lenses to focus the light. The GRIN lenses have usually a diameter of 0.5 mm. In this way, the GRIN lens can focus the light in the (laser) optical fiber into a spot, thereby increasing the luminous flux, that is, the light energy per unit area or light energy density, however, it results in an overlapping area of light and sound where only the photoacoustic signal can be detected to be restricted. Meanwhile, multiple optical elements lead to a longer rigid length (>10 mm) of the catheter, making it difficult for the catheter to pass through tiny arteries for intravascular endoscopy.

2. The optical element is located in the center of a ring UT, see Chinese Patent No. 201710364571.7 and U.S. Ser. No. 10/182,791B2.

In this method, the detection area is enlarged, but the use of the GRIN lens results in a larger UT, which leads to a larger catheter (diameter >1 mm). The diameter of the IVPA catheter should be limited to less than 1 mm to reduce the difficulty of passing through the artery. Most importantly, this design cannot be applied to intravascular endoscopes, because the large central aperture (corresponding to the GRIN lens) will reduce the performance of the UT.

There is a need to a solution to solve the problems in the prior art, especially considering that light is provided to make detection region not restricted and overlarge size of detector assembly/probe may be unsuitable for intravascular endoscopy in case of ultrasonic and photoacoustic dual-mode imaging.

### SUMMARY

This application particularly adopts photocurable glues and molds to manufacture a microlens integrated in an ultrasound transducer and adopts coaxial arrangement of devices to solve the problems of light supply and device dimensions (rigid length and diameter) and to simplify the manufacturing process for IVPA probe/catheter.

Provided herein is an ultrasound transducer assembly for ultrasonic and photoacoustic dual-mode imaging of an endoscope, the ultrasound transducer assembly comprising: an ultrasound transducer comprising an aperture; and a microlens integrated in the ultrasound transducer and used for collimating or focusing a light beam, wherein the microlens is accommodated in the aperture.

In certain embodiments, the microlens is formed by curing a photocurable glue in the aperture.

In certain embodiments, the photocurable glue is a quick-drying glue with high light transmittance.

In certain embodiments, the photocurable glue is an ultraviolet-curable glue.

In certain embodiments, the ultrasound transducer is a ring-type ultrasound transducer comprising a matching layer, a piezoelectric layer, a backing layer and electrode layers.

In certain embodiments, the microlens has a refractive index between 1.3 and 1.6.

In certain embodiments, the microlens has a diameter being less than 200 μm.

Provided herein is an ultrasonic and photoacoustic probe comprising: the ultrasound transducer assembly described above; a housing; a mirror; an optical fiber optically coupled with the microlens; a coil used for transmitting torque and adapted to be inserted into the housing to cause a scanning action of the ultrasonic and photoacoustic probe; and a wire connected to the ultrasound transducer to induce ultrasound; wherein the mirror, the ultrasound transducer assembly and the optical fiber are sequentially coaxially arranged in the housing.

In certain embodiments, the wire is connected to the ultrasound transducer by a silver glue.

In certain embodiments, the housing has a length being less than 3 mm and a diameter being less than 1 mm.

In certain embodiments, the ultrasonic and photoacoustic probe is used for ultrasonic and photoacoustic dual-mode imaging of an intravascular endoscope.

Provided herein is an endoscopy system comprising the ultrasonic and photoacoustic probe described above.

Provided herein is a method for manufacturing the ultrasound transducer assembly described above, the method comprising: providing a mold comprising a concave surface with a predetermined curvature; providing an ultrasound transducer comprising an aperture corresponding to the concave surface; placing the ultrasound transducer on the mold, wherein an axis of the aperture is aligned with an axis of the concave surface; introducing a photocurable glue into the aperture to fill a space within a side wall of the aperture and the concave surface; and curing the photocurable glue to form a microlens integrated in the aperture, wherein a curvature of the microlens is defined by the predetermined curvature of the concave surface.

In certain embodiments, the mold is a metal mold, and the concave surface with the predetermined curvature is formed on the mold by micro-machining of a computer numerically controlled machine.

In certain embodiments, the aperture is formed by laser micromachining.

In certain embodiments, the ultrasound transducer is a ring-type ultrasound transducer comprising a matching layer, a piezoelectric layer and a backing layer; and placing the ultrasound transducer on the mold includes attaching the matching layer to a surface of the mold where the concave surface is provided.

In certain embodiments, the microlens has a refractive index between 1.3 and 1.6.

In certain embodiments, the aperture has a diameter being less than 200 μm.

In certain embodiments, the photocurable glue is a quick-drying glue with high light transmittance.

In certain embodiments, the photocurable glue is an ultraviolet curable glue.

According to the present disclosure, a photocurable glue, especially an ultraviolet curable glue, is used to form a microlens integrated in the aperture of the ultrasound transducer, which is used to focus or collimate light in the coaxial design of IVPA probes/catheters, so that the light can be focused or collimated in a small space, the diameter of the micro-catheter/probe can be less than 1 mm, and the length can be less than 3 mm. In addition, the catheter/probe with microlens has a simple structure and a short rigid length, and can pass through blood vessels more safely for intravascular ultrasonic and photoacoustic dual-mode imaging or endoscopy. In addition, the coaxial design can obtain a large detection area (ultrasonic and photoacoustic overlapping area). Furthermore, the method for manufacturing the ultrasound transducer assembly proposed in the present application has a simple process and strong customization. The microlenses with different curvatures, refractive indices and other performances can be integrated into the ultrasound transducer assembly according to different needs while keeping other elements unchanged.

The application will be further explained below with reference to the drawings.

### BRIEF DESCRIPTION OF DRAWINGS

The accompanying drawings, in which the same reference numerals refer to the same or functionally similar elements, contain drawings of certain embodiments to further illustrate and clarify the various aspects, advantages and features disclosed herein. It will be understood that these drawings only depict certain embodiments of the invention and are not intended to limit its scope. The skilled person will understand that the elements in the figures are shown for simplicity and clarity and are not necessarily drawn to scale, wherein:

FIG. 1 shows an ultrasonic and photoacoustic probe including a UT assembly according to certain embodiments of the present invention;

FIG. 2 shows a ring-type UT according to certain embodiments of the present invention;

FIG. 3 shows a flow chart of manufacturing a UT assembly according to certain embodiments of the present invention; and

FIG. 4 shows two exemplary applications of the UT assembly according to certain embodiments of the present invention.

### DETAILED DESCRIPTION OF THE INVENTION

As used herein, the term “glue microlens” refers to an optical lens of less than 1 mm in diameter, and the optical lens is formed by curing a photocurable glue.

As mentioned above, the present application solves the problems of light supply and device dimensions (rigid length and diameter), especially by using a photocurable glue and mold to manufacture the microlens integrated in the ultrasound transducer and adopting the coaxial arrangement of the devices. The present application also simplifies the manufacturing process for IVPA probe/catheter. According to the present application, photocurable glues, particularly ultraviolet curable glues, are used to form microlenses, which can be used to focus or collimate light in the coaxial design of IVPA probes/catheters, so that light can be focused or collimated in a small space. The diameter of the micro-catheter/probe can be less than 1 mm (e.g., 0.5 mm-0.9 mm), and the length of the front-end hard tube can be less than 3 mm (e.g., 1.5 mm-2.5 mm). In addition, the catheter/probe with the microlens has a simple structure and a short rigid length, and thus can pass through micro blood vessels more safely for intravascular ultrasonic and photoacoustic dual-mode imaging or endoscopy. In addition, the coaxial design can get a large detection area (ultrasonic and photoacoustic overlapping area). Furthermore, the method for manufacturing the ultrasound transducer assembly provided in the present application involves a simple process and strong customization capability. Microlenses having different curvatures, refractive indices and other performances can be selected and integrated into the ultrasound transducer assembly according to different needs while keeping other elements unchanged.

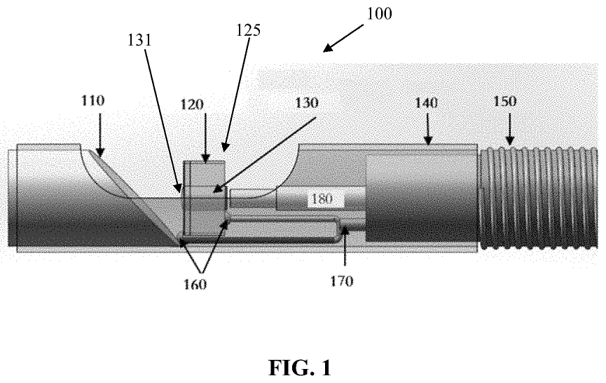

Referring to FIG. 1, it shows an ultrasonic and photoacoustic probe/catheter 100 including a UT assembly 125 according to certain embodiments of the present invention (the probe and the catheter can be used interchangeably herein). The probe/catheter 100 is particularly suitable for performing intravascular ultrasonic and photoacoustic dual-mode imaging.

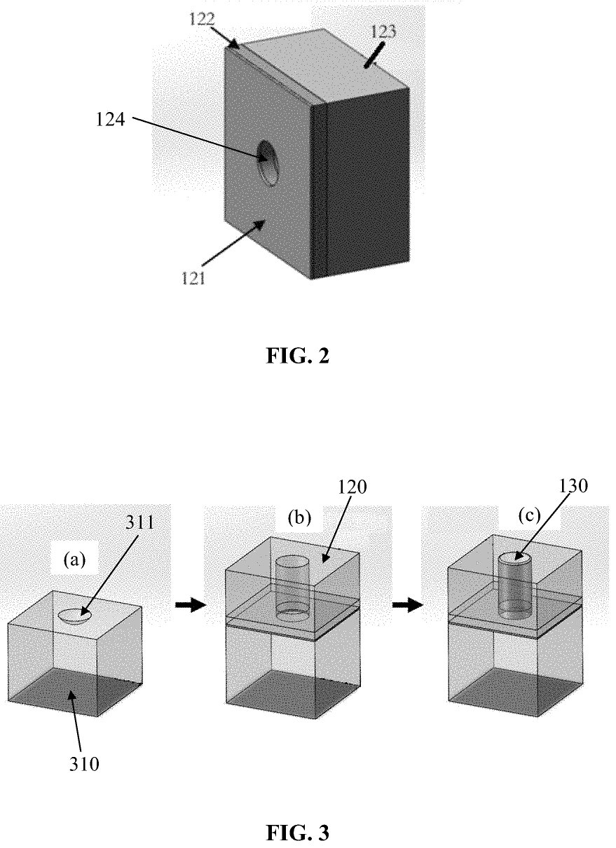

The catheter 100 includes a mirror 110, a ring-type UT 120, a glue microlens 130 that is received, accommodated or disposed in the ring-type UT 120, a housing 140, and a coil 150 (for example, torsion coils), wires 170 and optical fibers 180. The positive and negative electrodes of the wires 170 are connected to the UT 120 by a silver glue 160, or can be connected to the UT 120 by any means known in the art, for example, by applying voltage/current to the UT 120 to induce ultrasound (for example, pulses) to connect them together. The mirror 110 is placed at one end (terminal end) in the housing 140 for reflecting (collimated or focused) light beams (from the (laser) optical fiber optically coupled with the glue microlens 130) emitted from glue microlens 130. In particular, the UT assembly 125 includes the UT 120 and the glue microlens 130 integrated in the UT 120. The UT 120 has an aperture 124 (shown in FIG. 2) for allowing the light beam to pass through. The glue microlens 130 having a curved surface 131 is used to collimate or focus the light beam. The glue microlens 130 is integrated in the UT 120 in such a way that the glue microlens 130 is accommodated in the aperture 124 of the UT 120, fully fills the cross-section of the aperture 124 and adheres to the side wall of the aperture 124. The length of the housing 140 (or referred to as the front-end hard tube) can be limited to less than 3 mm, which is much shorter than that of the prior art. As shown in the figure, the UT 120 embedded with the glue microlens 130 is coaxially disposed with other elements, e.g., the mirror 110, the optical fiber 180 and the coil 150, which can lead to a large detection area. In particular, the glue microlens 130 is a photocured glue microlens that is formed by curing photocurable glue in the aperture 124 of the UT 120 and consists of the cured glue. More particularly, the photocurable glue is an ultraviolet curable glue, and thus the photocured glue microlens is an ultraviolet cured glue microlens. The coil 150 can induce the rotation and/or translation of the probe to perform imaging scanning.

As examples of photocurable glues, the Chinese application with application number 201410464946.3 discloses the formulation and properties of a liquid optical transparent glue, taking epoxy-terminated polysiloxane as the main composition of the liquid optical glue, which is applied to the bonding of transparent optical elements combined with UV light curing, and the disclosed refractive index is around 1.53. The refractive index of the optical glues disclosed in Chinese invention publications with application Nos. 201510341749.7, 201410300451.7 and a 201310328818.1 are generally around 1.50-1.53. The Chinese patent application with application No. 200810171323.1 discloses a high refractive index UV light curing coating glue for optical fiber coating, and its refractive index is in the range of 1.540-1.556. In addition, CN105802517A discloses a UV light curable glue whose refractive index is increased to more than 1.58, even to more than 1.60. In general, the present invention can use a quick-drying glue with high light transmittance, especially an ultraviolet curable quick-drying glue. The method disclosed in this application embeds/disposes the glue microlens in the UT, which can simplify manufacturing, provide customization capabilities, reduce the size of the element without reducing the performance of the UT (because only tiny holes or aperture is formed therein), and can form a coaxial configuration with other elements (including optical elements) to obtain a large detection/probing range.

In the present invention, it is preferable that the UT is a ring-type UT, which refers to a UT having a circular (micro) hole or aperture for receiving or accommodating a circular component. In particular, the ring-type UT does not have to have a circular/annular outer contour (a square/cuboid ring-type UT is shown in FIGS. 2-4 discussed below), as long as the UT has a circular hole or orifice for receiving or accommodating circular component (especially, lens, more particularly, micro-colloidal lenses, more particularly, micro-colloidal lenses, such as micro-colloidal lenses made from photocurable glue, especially by UV curable of UV glue). In a preferred embodiment of the present invention, the diameter of the orifice and therefore the formed micro-colloidal lens embedded therein is less than 200 μm, and the orifice and the micro-colloidal lens are very small. On the one hand, the entire device is miniaturized and facilitates application in narrow blood vessels. On the other hand, the small/micro orifice has little effect on the UT.

In certain embodiments, referring to FIG. 2, the ring-type UT 120 includes three layers: a matching layer 121, a piezoelectric layer 122, and a backing layer 123 (as shown in FIG. 2). The matching layer 121 is a layer that is in contact with/attached to the mold during the manufacturing process of the UT assembly. The piezoelectric layer 122 is a layer that performs piezoelectric action under an applied voltage/current to generate ultrasound for ultrasound or ultrasonic and photoacoustic dual-mode imaging. The backing layer 123 forms the backing of the device and is used to absorb the ultrasonic signal emitted backward. An aperture 124 is formed in the center of the UT 120 using laser micro-machining technology. The matching layer 121 faces the mirror 110. Ultraviolet photocurable glue is utilized to from the glue microlens at the center of the UT (e.g., in the central micro-hole or micro-aperture) for light focusing or collimation. The uncoated optical fiber is located in the center of the housing, and is aligned with the axis of the glue microlens. Wires are used to transfer signals (ultrasonic signals, for example, ultrasonic pulses) from the UT. In particular, its positive electrode is connected to the backing layer of the UT with silver glue, and its negative electrode is connected to the matching layer of the UT with silver glue. The positive and negative electrodes can be switched to each other. The coil (such as a torsion coil) is used to transmit torque for imaging scanning, and its end is suitable for insertion into the housing.

Referring to FIG. 3, it shows a flowchart of manufacturing a UT assembly according to certain embodiments of the present invention. The manufacturing method includes the following steps: providing a mold, where a smooth concave surface with a predetermined curvature is provided in the mold; providing a UT, and processing the UT to form an aperture corresponding to the smooth concave surface (e.g., the cross-sections of the aperture and the concave surface are the same); placing the UT on the mold, where the axis of the aperture of the UT is aligned with the axis of the smooth concave surface; introducing the photocurable glue into the aperture of the UT and make it fill the space within a side wall of the aperture of the UT and the smooth concave surface; and performing photocuring of the photocurable glue, thereby forming the glue microlens integrated in the aperture of the UT, adhering to the side wall and having a curved surface protruding from the aperture, where the curvature of the glue microlens is defined by a predetermined curvature of the smooth concave surface.

The light emitted by the laser (not shown) is easily scattered. Traditionally, people provide glass lenses or GRIN lenses to focus or collimate light, but their size is too large to fit into the micro-aperture (diameter less than 200 μm) of a UT suitable for intravascular endoscopy. In this context, the applicant proposes to combine a mold at a micro-aperture to cure the photocurable glue to form a glue microlens. Generally speaking, the refractive index of the ultraviolet curable glue used is between 1.3 and 1.6, which is similar to glass. The curvature of the glue microlens can be set with a mold. The curvature can be obtained by simulation of optical software or optical calculation under optical constraints as required, or can be specified.

Step (a) of FIG. 3 shows a mold 310 with a concave surface 311 for forming the glue microlens 130. The mold 310 is made of metal with smooth surfaces and is processed by numerically controlled machine tools. The mold 310 can be of different shapes (square, rectangular, circular, etc.), and the surface curvatures of the concave surface 311 can be different. The UT 120 is placed on the mold 310, and its matching layer 121 is adhered to the mold 310 (in step (b) of FIG. 3). The axis of the aperture 124 of the UT 120 is aligned with the axis of the concave surface 311 of the mold 310. Ultraviolet curable glue is used to fill the space within the aperture 124 and the concave surface 311. The glue microlens 130 is formed under irradiation of ultraviolet light (in step (c) of FIG. 3). It is easy to understand that the mold and the concave surface of the mold can be processed by any technology understood by those skilled in the art to have the curvature and size obtained by simulation or calculation. The curvature and size of the concave surface of the mold define or correspond to or equal to the curvature and size of the formed glue microlens. In addition, the type and specific composition of the photocurable glue (preferably, the ultraviolet curable glue with high light transmittance) can be selected according to needs to obtain the desired refractive index and other properties. Some applications/patents mentioned above give some examples of photocurable glues. Those skilled in the art can easily understand these and other curable glues and their curing conditions and processes, and the applicant will not repeat them here.

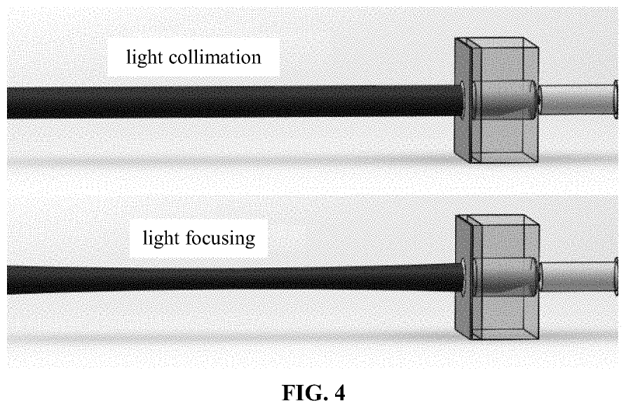

FIG. 4 shows an exemplary application of the UT assembly according to certain embodiments of the present invention. According to actual needs, by customizing the radius of curvature (curved surface of the mold and therefore the glue microlens formed by the curved surface) or/and the distance between the glue microlens and the optical fiber, the light from the optical fiber (e.g., laser beam) can be passed through the glue microlens as shown in FIG. 4 for light collimation or light focusing. The present invention provides customization capabilities, which can customize or re-manufacture the glue microlens integrated with the UT according to the required specifications or other requirements without changing other elements (by replacing the mold or selecting other photocurable glues). In addition, the probe or catheter of the present application can be used with other components of a conventional intravascular ultrasonic and photoacoustic endoscopy system to form a new type of intravascular ultrasonic and photoacoustic endoscopy system. For example, the endoscopy system can include a light source (e.g., pulsed laser light source), controller, signal acquisition unit, signal analysis unit, etc., and can complete the application of voltage/current, signal acquisition, signal analysis, etc., based on ultrasonic and photoacoustic dual-mode imaging.

As described above, the present disclosure specifically addresses the problems of the light supply and device dimensions (rigid length and diameter) by using photocurable glues and molds to manufacture the microlens integrated in the UT and adopting the coaxial arrangement of the devices, and this application simplifies the manufacturing process of IVPA probe/catheter. According to this application, photocurable glues, especially ultraviolet curable glues, are used to form the microlens, which is used to focus or collimate light in the coaxial design of IVPA probes/catheters, so that the light can be focused or collimated in a small space. The diameter of the micro-catheter/probe can be less than 1 mm, and the length can be less than 3 mm. In addition, the catheter/probe with microlens has a simple structure and a short rigid length, and can pass through blood vessels more safely for intravascular ultrasonic and photoacoustic dual-mode imaging or endoscopy. In addition, the coaxial design can obtain a large detection area (ultrasonic and photoacoustic overlapping area). Furthermore, the method for manufacturing the UT assembly proposed in the present application is simple and has strong customization capability. The microlenses with different curvatures, refractive indices and other performances can be integrated into the UT assembly according to different needs while keeping other elements unchanged.

Those skilled in the art can understand that various changes and/or modifications can be made to the present invention illustrated in the embodiments without departing from the spirit or scope of the present invention as broadly described. Therefore, these embodiments are considered to be illustrative rather than restrictive in all aspects.

What is claimed is:

1. An ultrasound transducer assembly for ultrasonic and photoacoustic dual-mode imaging of an endoscope, the ultrasound transducer assembly comprising:
an ultrasound transducer comprising an aperture; and
a microlens integrated in the ultrasound transducer and used for collimating or focusing a light beam, wherein the microlens is accommodated in the aperture.

2. The ultrasound transducer assembly according to claim 1, wherein the microlens is formed by curing a photocurable glue in the aperture.

3. The ultrasound transducer assembly according to claim 2, wherein the photocurable glue is a quick-drying glue with high light transmittance.

4. The ultrasound transducer assembly according to claim 2, wherein the photocurable glue is an ultraviolet-curable glue.

5. The ultrasound transducer assembly according to claim 1, wherein the ultrasound transducer is a ring-type ultrasound transducer comprising a matching layer, a piezoelectric layer, a backing layer and electrode layers.

6. The ultrasound transducer assembly according to claim 1, wherein the microlens has a refractive index between 1.3 and 1.6.

7. The ultrasound transducer assembly according to claim 1, wherein the microlens has a diameter being less than 200 μm.

8. An ultrasonic and photoacoustic probe comprising:
the ultrasound transducer assembly according to claim 1;
a housing;
a mirror;
an optical fiber optically coupled with the microlens;
a coil used for transmitting torque and adapted to be inserted into the housing to cause a scanning action of the ultrasonic and photoacoustic probe; and
a wire connected to the ultrasound transducer to induce ultrasound;
wherein the mirror, the ultrasound transducer assembly and the optical fiber are sequentially coaxially arranged in the housing.

9. The ultrasonic and photoacoustic probe according to claim 8, wherein the wire is connected to the ultrasound transducer by a silver glue.

10. The ultrasonic and photoacoustic probe according to claim 8, wherein the housing has a length being less than 3 mm and a diameter being less than 1 mm.

11. The ultrasonic and photoacoustic probe according to claim 8, wherein the ultrasonic and photoacoustic probe is used for ultrasonic and photoacoustic dual-mode imaging of an intravascular endoscope.

12. An endoscopy system comprising the ultrasonic and photoacoustic probe according to claim 8.

13. A method for manufacturing the ultrasound transducer assembly according to claim 1, the method comprising:
providing a mold comprising a concave surface with a predetermined curvature;
providing an ultrasound transducer comprising an aperture corresponding to the concave surface;
placing the ultrasound transducer on the mold, wherein an axis of the aperture is aligned with an axis of the concave surface;
introducing a photocurable glue into the aperture to fill a space within a side wall of the aperture and the concave surface; and
curing the photocurable glue to form a microlens integrated in the aperture, wherein a curvature of the microlens is defined by the predetermined curvature of the concave surface.

14. The method according to claim 13, wherein the mold is a metal mold, and the concave surface with the predetermined curvature is formed on the mold by micro-machining of a computer numerically controlled machine.

15. The method according to claim 13, wherein the aperture is formed by laser micromachining.

16. The method according to claim 13, wherein the ultrasound transducer is a ring-type ultrasound transducer comprising a matching layer, a piezoelectric layer and a backing layer; and placing the ultrasound transducer on the mold includes attaching the matching layer to a surface of the mold where the concave surface is provided.

17. The method according to claim 13, wherein the microlens has a refractive index between 1.3 and 1.6.

18. The method according to claim 13, wherein the aperture has a diameter being less than 200 μm.

19. The method according to claim 13, wherein the photocurable glue is a quick-drying glue with high light transmittance.

20. The method according to claim 13, wherein the photocurable glue is an ultraviolet curable glue.
